SEQUENCE LISTING (4 sequences)

SEQ ID NO: 1 - protein, 290 aa, Neurospora crassa
MAGLHSRLTTFLLLLLSALPAIAAAAPSSGCGKGPTLRNGQTVTTNINGKSRRYTVRLPDNYNQNNPYRLIFLWHPLGSSMQKIIQGEDPNRGGVLPYYGLPPLDTSKSAIYVVPDGLNAGWANQNGEDVSFFDNILQTVSDGLCIDTNLVFSTGFSYGGGMSFSLACSRANKVRAVAVISGAQLSGCAGGNDPVAYYAQHGTSDGVLNVAMGRQLRDRFVRNNGCQPANGEVQPGSGGRSTRVEYQGCQQGKDVVWVVHGGDHNPSQRDPGQNDPFAPRNTWEFFSRFN

SEQ ID NO: 2 - DNA, 873 bp, Neurospora crassa
atggcaggccttcactcccgcctcaccacttttctccttctcctcctctctgccctccccgccatcgccgccgccgccccctcctccggctgcggtaaaggtcccaccctccgcaacggccaaaccgtcaccaccaacatcaacggcaaatcgcgtcgctacaccgtccggcttccggacaactacaaccaaaacaacccctaccgcctcatcttcctctggcaccccctcggcagctccatgcaaaagatcattcagggcgaagaccccaaccgcggcggcgtcctgccttactacggcctgccgcccctcgacacgagcaagagtgccatctacgtggttccggacgggttgaacgcgggctgggcgaaccagaacggggaggacgtctcctttttcgacaacattctgcaaaccgtgagcgacgggctgtgcatcgacaccaacctcgtcttcagcacgggcttctcctacggcggcggcatgagtttcagcctggcgtgctcgcgggcgaacaaagtccgtgccgtcgctgtcatctctggcgcgcagctttctggctgcgcgggcgggaatgatccggtggcgtactacgcgcaacacggaacgtccgacggcgtgctgaatgtggcgatgggaagacagttgagagatcggttcgtgaggaacaatgggtgtcagccggcaaacggggaagtgcagcctggtagtgggggacggtctacgagggttgagtatcagggatgtcaacagggtaaggacgtggtgtgggtggtgcatggcggagatcataatccgagtcagagggatccgggacagaacgacccgtttgcgccgaggaacacttgggagttctttagcaggttcaactag

SEQ ID NO: 3 - protein, 530 aa, Talaromyces stipitatus
MMLTSAILLLTLGVQLSHADDSSRENFSNRCDQLAKEIHIPNVTVNFVEYVANGTNVTLADNPPSCGQSNQVVLADLCRVAMEVTTSNQSQITLEAWFPENYTGRFLSTGNGGLAGCIQYVDMAYASSMGFATVGANGGHNGTSGESFYHNPDIVEDLSWRSVHTGVVVGKELTKKFYHEGFHKSYYLGCSTGGRQGFKAVQEFVHDFDGVVAGCPAFNFVNLNSWSGHFYPITGNSSADTFLTTAQWTLVQQSVMEQCDSLDGAVDGVIEAIDQCHPVFEQLICRPGQNASECLTGKQVNTAQLVLSPIYGTKGEFLYPRMQPGVENVDMYITYNGDPFAYSTDWYKYVVFSDPNWDPATLNAQDYEIALAQNPSNIQTFEGDLSAFRDAGAKVLTYHGTADPIITGETSKVYYRHVAETMNAAPEELDEFYRYFRIGGMSHCGGGTGATAIGNVLSAQWSNDPDANVLMAMVRWVEEGVAPEYIRGASLGSGPGAKVEYTRRHCKYPTRNVYVGPGNWTDENAWKCIL

SEQ ID NO: 4 - DNA, 1900 bp, Talaromyces stipitatus
gcttttttcttggctttttcttccactatgcttcatcgcgtgggagaagcaaaacgaatccgtgaacatccgagatgatgttgacaagtgcaatcctcctcctgactctcggggtccagctgagtcacgcagatgattcgtcccgcgagaacttctcaaatcgatgcgaccagctggctaaggagatccatattcccaacgtaactgtaaactttgtggaatatgttgccaatggcaccaacgtcacacttgctgacaatccgccttcatgcgggcagtccaatcaagtggtcctggcggatttgtgtcgagtggctatggaggttacaacctccaaccagagtcagatcaccttggaggcatggtttccggaaaactacactggaaggtttctgagtaccggtaatggtggtctcgcagggtgtcagttccttccctcccctatcttggtggtgtgcattgcggtgaaacaaattaacaccatcacccggagattaggtatccagtatgtagacatggcatatgcgtcgtcaatgggatttgcgacagtaggcgcaaatgggggccacaatggcacctccggagaatccttctaccacaacccggacatcgtagaggatctctcttggcgatcagtgcataccggtgtcgtcgtgggcaaagaattgaccaaaaaattctaccacgaaggattccacaaatcatactatctcggctgctctacaggcggacgacaagggttcaaggcagtccaagaatttgtgcatgactttgacggcgtggtcgcagggtgtccggcgttcaactttgtaaatctcaacagctggagtggacatttctaccctatcacaggcaactcatccgcagacacgttcctgacaaccgcacagtggacactagtacaacaatcggttatggagcaatgtgattctctcgatggcgcggttgacggcgtaattgaagctatcgatcaatgccacccggtctttgagcaactcatctgcagaccgggacaaaatgcttcggaatgtctgacaggaaagcaagtcaataccgcccaactcgtcctttccccaatctacggaacaaaaggagaattcctctatccgcgcatgcagcccggagtagaaaatgtcgacatgtacataacctacaacggtgacccattcgcctacagcaccgactggtacaaatacgtcgttttcagcgatccaaattgggatcccgcaaccttgaacgcgcaggattacgagattgcgcttgctcaaaacccgtccaatatccagacatttgagggtgatttatccgctttccgcgatgcaggagctaaagtcttgacctatcacggcactgcagacccgattatcacgggggagacgtcaaaggtatactaccgtcacgtcgctgagaccatgaacgcagctccagaggaattagatgagttttatcgctatttccggattggaggtatgagccactgcggtggaggcacgggagccacggcgattggtaatgtgctcagtgcgcaatggagcaatgatcctgacgctaatgtgttgatggcgatggtacgctgggttgaggaaggggttgctccggagtatattcgtggtgcttcgcttggtagtgggccgggagcaaaggttgagtatactcggcggcattgcaagtatccgacgaggaatgtttatgttgggcctggaaattggacggacgagaatgcgtggaaatgtattttgtagtagcttcatcaagcttgcagcggattcttcgagagcagggggctggagcatgaaaaggtgtatactagcttgagccaggcactaattgattctaccgtcaagggggggagatgcgggaaagttatacggacgtttaagggctgcgagacattcctgaa

(19) United States
(12) Patent Application Publication
(10) Pub. No.: US 2010/0256353 A1
(43) Pub. Date: Oct. 7, 2010

(54) USE OF TYPE C AND D FERULOYL ESTERASES IN THE MANUFACTURE OF BIOFUELS

(75) Inventors: Stuart Ian West, Wales (GB); Hadyn Gregg William, Wales (GB)

Correspondence Address: K&L Gates LLP, 1900 MAIN STREET, SUITE 600, IRVINE, CA, 92614-7319, US

(73) Assignee: BIOCATALYSTS LTD., PARC Nantgarw (GB)

(21) Appl. No.: 12/675,691

(22) PCT Filed: Aug. 21, 2008
(86) PCT No.: PCT/GB2008/002844

§ 371 Date: Mar. 1, 2010

(30) Foreign Application Priority Data
Aug. 28, 2007 (GB) 0716702.6

(51) Int. Cl. C10L 1/18; C12P 1/00; C12P 19/00; C12P 19/22; C12P 19/20

(52) U.S. Cl. 536/123; 435/41; 435/72; 435/95; 435/96

(57) ABSTRACT

The use of an enzyme preparation comprising type C feruloyl esterase or type D feruloyl esterase in the manufacture of biofuels from plant cell wall materials. A method for manufacturing biofuels from plant cell wall materials by converting lignocellulosic materials in said plant cell walls to sugars suitable for use as a fermentation feedstock, which method comprises (i) contacting said plant cell wall material with an enzyme preparation comprising type C feruloyl esterase or type D feruloyl esterase and plant cell wall degrading enzymes and (ii) separating any soluble sugars therefrom for bioconversion to biofuel. A slurry prepared by converting lignocellulosic materials in plant cell walls to sugars using an enzyme preparation comprising type C feruloyl esterase or type D feruloyl esterase, and optionally plant cell-wall degrading enzymes.

The present invention relates to the use of enzyme preparations with activity against a broad range of hydroxycinnamic acid esters to release soluble sugars from plant materials and which soluble sugars may be utilised in the production of biofuels.

### BACKGROUND AND PRIOR ART

Plant cell walls contain phenolic acid residues which are ester linked to the polysaccharide network. The most abundant of these phenolic residues is ferulic acid. However, p-coumaric acid and other hydroxycinnamic acids are also common in most plants.

Ferulic acid has been shown to cross link hemicellulose and lignin (Ralph et al., 1995). The presence of these hydroxycinnamic acids linked to sugars can act as a significant barrier to enzymatic hydrolysis unless they are removed.

Certain microorganisms, have however, evolved enzymes, such as feruloyl esterase enzymes (E.C. 3.1.1.73) also known as ferulic acid esterases, that are able to breakdown lignocellulosic feedstocks by breaking the ester bond between the phenolic residue and cell wall polysaccharides (Williamson et al., 1998). Once the ester bond has been hydrolysed the polysaccharide backbone then becomes accessible to other lignocellulolytic enzymes. Filamentous fungi, such as Aspergillus niger, are well known producers of plant cell wall degrading enzymes.

Feruloyl esterases were originally classified into two groups based on their specificity for different hydroxycinnamic acids. Type A feruloyl esterases, such as feruloyl esterase A of Aspergillus niger (AnFAEA) are active against ferulate, sinapate and p-courmarate, but not caffeate. They are also capable of releasing 5-5′ and 9-O-4′ ferulate dehydrodimers from plant material. Type B feruloyl esterases such as feruloyl esterase B of Aspergillus niger (AnFAEB) are active against esters of ferulate, caffeate and p-courmarate, but not sinapate or ferulate dehydrodimers.

Two additional classes of feruloyl esterases with broader substrate specificity were proposed by Crepin et al., (2004a). Type C feruloyl esterases such as feruloyl esterase C of Talaromyces stipatatus (TsFAEC) are active against esters of ferulate, caffeate, p-courmarate and sinapate but not ferulate dehydrodimers. Type D feruloyl esterases such as Psudomonas fluorescences XYLD are active against esters of ferulate, caffeate, p-courmarate, sinapate and ferulate dehydrodimers. Type A and type B feruloyl esterases have previously been used to help break down plant to sugar solutions.

Treatment of corn stover and leaves with a type B ferulic acid esterase preparation from Humicola insolens, which released ferulate and courmarate, before treatment with cellulases has been shown to significantly increase breakdown of the corn stover and increase the release of sugars and phenolics, Akin et al., (2006). Furthermore, Tabka et al., (2006) described a combined treatment using combinations of cellulose and xylanase from Trichoderma reesei, laccases from Pycnoporus cinnabarinus and a type A recombinant feruloyl esterase from Aspergillus niger (AnFAEA) to convert wheat straw into fermentable sugars. A synergistic effect between cellulases, FAE and xylanases was proven under a critical enzymatic concentration (10 U/g of cellulase, 3 U/g of xylanase and 10 U/g of FAE).

GB 2324302 describes a phenolic acid esterase with ferulic acid esterase and coumaric acid esterase activity, and suggests that it can be used to improve the digestibility of lignocellulose waste to sugars. However, the inventors do not describe any activity of the enzyme against caffeate, sinapate or ferulate dehydrodimers.

Recombinant fusion proteins in which the sequences for feruloyl esterase A of Aspergillus niger (FAEA) and xylanase B (XYNB) are combined have been used to degrade plant cell wall material. In EP 1752533, the authors have suggested that these fusion proteins could have application in bioethanol manufacture and the bleaching of pulp and paper. However, the enzyme combinations recommended did not show broad specificity against a range of hydroxycinnamic acid esters.

The use of enzymes to break down lignocellulosic materials into fermentable sugars has been extensively studied in recent years. Cellulase preparations in isolation will not release all of the available sugars. To obtain an optimum yield of sugars the process requires a cocktail of enzymes with activities against a wide variety of carbohydrates to break down the complex interwoven carbohydrate polymers that form the plant cell wall. Various enzyme mixtures have been suggested as providing increased yield of sugars.

For example, US 2004/0005674 describes a method for generating free sugars and oligosaccharides from lignocellulosic biomass using cellulases, xylanases, ligninases, amylases, proteases, lipases and glucuronidases. U.S. Pat. No. 5,882,905 describes how a thermostable alpha-L-furanosidase can be used in conjunction with xylanolytic enzymes for the treatment of hemicellulosic materials to produce fermentable sugars. US 2007/0077630 describes the use of a cellulolytic protein in combination with a polypeptide having cellulolytic enhancing activity to degrade lignocellulose.

However, none of these documents disclose a combination of enzymes that will release substantially all of the phenolic residues in plant material.

The US ethanol industry produces more than 3.4 billion gallons of ethanol a year, approximately 95% of which was derived from corn starch (US Department of Agriculture, 2002). Corn starch can be readily converted to fermentable sugars, but, it is also a valuable food product. Further, as the cost of corn is a major factor in the economics of bioethanol manufacture, there are significant benefits to using cheaper alternative carbohydrate containing feedstocks such as grass, corn stover, wheat straw, spent grain, bran and waste paper etc., which are renewable, readily available and many of which are currently treated as waste materials. These can be converted to sugars by treatment with appropriate enzymes. For example, WO 96/37627 describes a process for producing ethanol from vegetable material using enzymes capable of degrading cellulose, hemicellulose and starch.

WO 1994/029474 describes a process for producing ethanol from cellulosic biomass involving the addition of a cellulase enzyme to biomass added directly to a fermentation. The enzyme helped to breakdown the cellulosic biomass to sugars allowing it to be used as a source of carbon for the fermentation (a technique known as simultaneous saccharification and fermentation).

However, the lignocellulose in these materials is much more difficult to convert to fermentable sugars than starch. Pre-treatment of the lignocellulose is usually required before it can be efficiently converted to ethanol (McMillian, 1994). For example, Martin et al., (2002) described a method for producing ethanol from sugarcane bagasse using steam explosion and treatment with laccases followed by fermentation with a recombinant xylose utilising Saccharomyces cervisae.

There is considerable interest in the use of plant materials rich in lignocellulose, as a feedstock for the production of bioethanol. However, hydroxycinnamic acids ester-linked to the carbohydrates in lignocellulose can act as a significant barrier to enzymatic hydrolysis, and the inability of the enzymes currently used to remove certain hydroxycinnamic acids can significantly limit the yield of sugars extracted.

The present inventors have sought to alleviate the deficiences in the prior art and utilised feruloyl esterase preparations that are active against a broad range of hydroxycinnamic acid esters in a process to extract fermentable sugars in a high yield from lignocellulose substrates derived from plant cell walls and which can be utilised in the efficient production of biofuel.

### SUMMARY OF THE INVENTION

Therefore, according to a first aspect of the present invention, there is provided use of an enzyme preparation comprising type C feruloyl esterase or type D feruloyl esterase, in the manufacture of biofuels from lignocellulose derived from plant cell walls.

The use of broad spectrum type C and type D feruloyl esterase in the production of biofuels such as bioethanol has not been previously suggested. Advantageously, the broad spectrum activity of these feruloyl esterases against hydroxycinnamic acids, facilitates extraction of high yields of fermentable sugars which may be utlilised in the production of biofuels. The limited substrate range of the individual feruloyl esterase enzymes that have previously been used limits the breakdown of the plant cell wall material containing hydroxycinnamic acids and renders the plant cell-wall degrading enzymes inactive. These hydroxycinnamic acid residues, therefore, act as a physical barrier preventing further breakdown of the plant cell wall polysaccharides by plant cell wall degrading enzymes, such as xylanases and cellulases. The feruloyl esterases according to the present invention have been found to be particularly broad spectrum, thus removing a larger number of hydroxycinnamic acids and substantially reducing the physical barrier that these acids present to other enzymes utilised to breakdown plant cell wall material.

To obtain a high yield of sugars from lignocellulosic feed stocks such as grasses, corn stover, wheat stalks, brewers spent grain, waste paper, etc., it is necessary to use a cocktail of enzymes with broad substrate specificity. Therefore, in a preferred embodiment of this aspect of the invention, the enzyme preparation comprises plant cell wall degrading enzymes, including cellulases and xylanases. Other appropriate enzymes may also be included in the preparation, for example, laccase, pectinase, glucanase, mannanase, amylase and arabinofuranosidase.

The present invention, therefore, provides a higher yield of soluble sugars by ensuring that a broader variety of the hydroxycinnamic acid residues are released, opening up the structure to further degradation by other enzymes and increasing the resulting yield of soluble sugars.

Therefore the higher yields of soluble sugars obtained using the specified feruloyl esterases also advantageously allows the efficient production of biofuels when these feruloyl esterases are combined with appropriate plant cell wall degrading enzymes.

Therefore, according to a further aspect of the invention, there is provided, a method for manufacturing biofuels from plant cell wall materials by converting lignocellulosic materials in said plant cell walls to sugars suitable for use as a fermentation feedstock, which method comprises (i) contacting said plant cell wall material with an enzyme preparation comprising type C feruloyl esterase and/or type D feruloyl esterase and plant cell-wall degrading enzymes and (ii) separating any soluble sugars therefrom for conversion to biofuel.

Plant cell-wall degrading enzymes refers to enzymes that are able to digest the cell-wall components, such as cellulose, hemicellulose and lignin.

According to an even further aspect of the invention there is provided a slurry prepared by converting lignocellulosic materials in plant cell walls to sugars using an enzyme preparation comprising type C feruloyl esterase or type D feruloyl esterase, and optionally plant cell-wall degrading enzymes.

### DETAILED DESCRIPTION OF THE INVENTION

In a preferred embodiment of the invention, the plant cell wall degrading enzymes used in the enzyme preparation utilised comprise cellulases and/or xylanases and also may preferably include additional enzymes including laccase, pectinase, glucanase, mannanase, amylase and arabinofuranosidase. The plant material containing lignocellulose may, therefore, be hydrolysed with a cocktail of enzymes including cellulases, hemicellulases, such as xylanase and a feruloyl esterase or mixture of feruloyl esterase preparations that provides activity against esters of ferulate, caffeate, p-courmarate, sinapate and ferulate dehydrodimers. The resulting hydrolysate is rich in soluble sugars and can be used as a feedstock for bioethanol production. Suitable cellulases include cellobiodydrolases, endoglucanases, exoglucanases or glucosidases.

Feruloyl esterases can exhibit synergy with specific xylanases. For example Faulds et al., 2003 demonstrated that in general family II xylanases were preferred synergistic partners with feruloyl esterases for the release of ferulate. A type C feruloyl esterase from Taloromyces stiputatus (TsFAEC) was shown to release 100% of the ferulic acid from water extractable wheat endosperm arabinoxylan, when acting in combination with a xylanase from Trichoderma longibrachiatum. Hence, the choice of xylanase to maximise sugar release may be critical.

Further, many of the enzymes required for the breakdown of lignocellulose are produced at low levels by native organisms making the enzymes prohibitively expensive to manufacture. High levels of expression of the required enzymes can be achieved in recombinant microbial expression systems. This allows for more efficient production of the required enzymes. For example, US 2005/0191736 and US 2005/0233423 describe several recombinant polypeptides and their application in breaking down lignocellulosic materials including corn stover.

Examples of type C and type D feruloyl esterases have been cloned into yeast high expression systems, and are now available for more cost effective production of these enzymes.

For example, a novel type C feruloyl esterase from Taloromyces stiputatus is described in WO 2004/009804 as having a broad spectrum of hydrolytic activity against esters of hydroxycinnamic acids has been cloned into a Pichia pastoris strain capable of high levels of expression. (Crepin et al., 2003).

Similarly, Crepin et al., (2004b) identified a novel type D feruloyl esterase from Neurospora crassa, and cloned and expressed the corresponding recombinant protein in Pichia pastoris. This recombinant enzyme was highly active against esters of ferulate, caffeate, p-courmarate, sinapate and ferulate dehydrodimers.

A preferred embodiment of all aspects of the present invention uses recombinant type C and/or type D feruloyl esterases preferably in combination with cellulases and xylanases and which advantageously provides a more cost effective process with a high yield of fermentable sugars from lignocellulosic feedstocks.

In a preferred embodiment of the invention a recombinant type C feruloyl esterase from Talaromyces stipatatus (TsFAEC) and a recombinant type D feruloyl esterase from Neurospora crassa (NsFAED), which are active against a broader range of hydroxycinnamic acids than the type A or type B feruloyl esterase enzymes are used because they can be produced in high quantities and are more cost effective than the wild type enzymes.

The lignocellulosic substrate can be chosen from one or more of the following materials, which is not exhaustive and is intended only to provide examples of materials that can be used.

The lignocellulosic material may be derived from plant stems and leaves including, grass, corn stover and wheat stalks. Fruit tissues and vegetable tissues including potato peelings, carrot peelings may also be used as well as fibre from cereals including wheat bran, corn germ and oat bran. Other sources include residues from fruit or vegetable juice extraction such as apple pomace, pear pomace. berry pomace, the residue from carrot juice extraction, residues from crushing seeds including palm meal, rape meal, sunflower meal, orujo (olive waste). Waste materials including waste paper, waste foods, sewage solids may also be used.

The lignocellulosic material should be sorted or washed to remove inert undigestable material such as dirt, stones and chemical residues.

The lignocellulosic material is preferably initially chopped, ground or milled to a diameter of less than 4 cm. The preferred particle size is between 3 mm and 5 mm and even more preferably 4 mm. Water or buffer may be added to form a slurry with a moisture content of from 30% and 95% w/w. The preferred moisture content of the slurry is from 60% to 80%, but preferably 70%.

The pH of the slurry should be preferably from pH 3.5 and pH 8.5. The preferred pH of the slurry is from pH 4.0 and pH 6.0 and even more preferably 5.0. Acids including hydrochloric acid, sulphuric acid, phosphoric acid or buffers may be used in place of water to bring the pH into the desired range.

The slurry of lignocellulosic material may be pre-warmed or heated to from 20° C. to 100° C. for up to 1 hour to ensure the material is properly wetted. This step is particularly useful for materials such as grass which are highly lignified and difficult to wet.

Cellulase and xylanase enzymes may then be added to the slurry. Preferably from 30,000 units of cellulase from Trichoderma sp. to 150,000 units of xylanse from Trichoderma sp. per kg dry weight of lignocellulose is used.

One unit of cellulase activity is defined as that amount of enzyme that causes the release of 1.25 micromoles of glucose equivalents per minute at pH 4.6 and 40° C. from a solution of carboxy methyl cellulose. The cellulase activity of enzyme preparations can be confirmed by comparing them using cellulase assay procedure 17 (available from Biocatalysts Ltd., Wales, United Kingdom) to a sample of Cellulase 13L from Biocatalysts Ltd which has a specified cellulase activity of 1,500 units per gram. One unit of xylanase activity is defined as that amount of enzyme that causes the release of 1 micromole of xylose equivalents per minute at pH 4.6 and 40° C. from a solution of oat spelt xylan. The xylanase activity of enzyme preparations can be confirmed by comparing them using xylanase assay procedure 55 (available from Biocatalysts Ltd.) to a sample of Depol 333L from Biocatalysts Ltd which has a specified xylanase activity of 11,000 units per gram.

Examples of commercial enzyme preparations with cellulase or xylanase activities that can be used include Cellulase 13L, Cellulase 13P, Depol 112L, Depol 40L, Depol 692L, Depol 761 P, Depol 762P (from Biocatalysts Ltd.), Cellulclast, Celluzyme, Cereflo, Ultraflo, Novozym 188 (from Novozymes AS), Laminex and Spezyme CP (from Genencor).

Feruloyl esterase preparations or mixtures of feruloyl esterase preparations with activity against esters of ferulate, caffeate, p-courmarate, sinapate and ferulate dehydrodimers may be added to the slurry. The activity of the feruloyl esterase preparation against methyl ferulate is preferably between 50 to 5000 units per kg dry weight of lignocellulosic material. Preferably, the activity of the methyl ferulate preparation against methyl ferulate will be 1000 units per kg dry weight of lignocellulosic material,

One unit of feruloyl esterase activity is defined as that amount of enzyme that causes the release of 1.0 micromole of ferulic acid per minute at pH 6.0 and 37° C. from a solution of methyl ferulate. The feruloyl esterase activity of enzyme preparations can be confirmed by comparing the feruloyl esterase activity using the feruloly esterase assay procedure 24 (available from Biocatalysts Ltd.) to a sample of Depol 740L from Biocatalysts Ltd., which has a specified feruloyl esterase activity of 36 units per gram.

Preferably, the feruloyl esterase to be utilised according to the invention is a type D feruloyl esterase.

The feruloyl esterase may preferably be in a mixture of type C feruloyl esterase and type D feruloyl esterase. Alternatively, the feruloyl esterase may be included in a mixture of type B feruloyl esterase and type D feruloyl esterase.

En another embodiment of the invention the feruloyl esterase may be a type C feruloyl esterase and which may be included in a mixture of type A feruloyl esterase and type C feruloyl esterase.

A preferred embodiment of the invention is for the feruloyl esterase to be a recombinant feruloyl esterase.

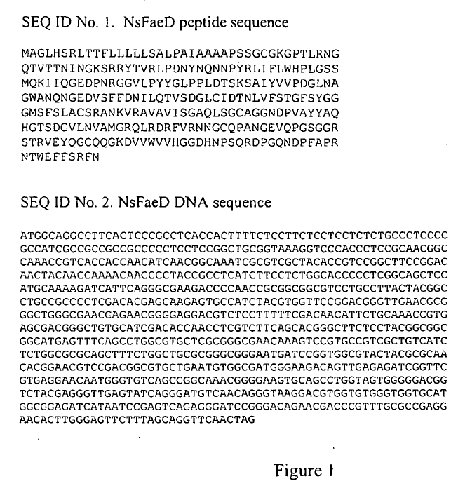

A preferred embodiment of the invention is for the feruloyl esterase to be recombinant type D feruloyl esterase and preferably a type D feruloyl esterase from Neurospora crassa (NsFAED) and preferably with a protein sequence (SEQ ID NO. 1) as shown in FIG. 1, or a recombinant polypeptide with feruloyl esterase activity and exhibiting from 80% homology to NsFAED (SEQ ID NO. 1). The DNA sequence encoding for NsFAED is shown in FIG. 1 (SEQ ID NO. 2).

A preferred embodiment of the invention is for the feruloyl esterase to be a mixture of recombinant type C feruloyl esterase and recombinant type D feruloyl esterase.

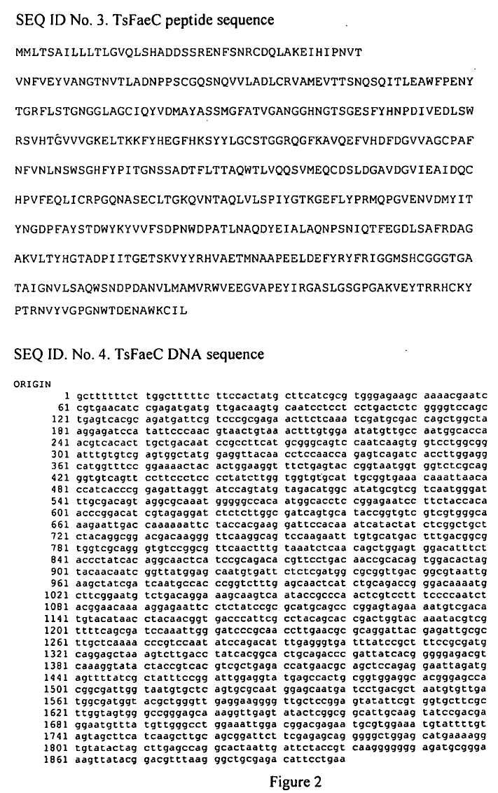

The feruloyl esterase may preferably be a mixture of recombinant type C feruloyl esterase from Taloromyces stiputatus (TsFAEC) with a sequence as shown in FIG. 2 (SEQ ID NO. 3) or a recombinant polypeptide with feruloyl esterase activity and exhibiting from 80% homology to TsFAEC (SEQ ID NO. 3), and a recombinant type D feruloyl esterase from Neurospora crassa (NsFAED) with a sequence as shown in FIG. 1, or a recombinant polypeptide with feruloyl esterase activity and exhibiting from 80% homology to NsFAED (SEQ ID NO. 1). The DNA sequence encoding for TsFAEC is shown in FIG. 2 (SEQ ID NO. 4).

A preferred embodiment of the invention is for the feruloyl esterase to be a mixture of type B feruloyl esterase and a recombinant type D feruloyl esterase from Neurospora crassa (NsFAED) with a sequence as shown in FIG. 1, or a recombinant polypeptide with feruloyl esterase activity and exhibiting from 80% homology to NsFAED (SEQ ID NO. 1).

Homology refers to sequence similarity between sequences and can be determined by comparing a position in each sequence which may be aligned for purposes of comparison. When a position in the compared sequence is occupied by the same nucleotide base or amino acid, then the molecules are homologous at that position. A degree of homology between sequences is a function of the number of matching or homologous positions shared by the sequences.

“Percent (%) amino acid sequence identity” with respect to the polypeptide sequences identified herein is defined as the percentage of amino acid residues in a candidate sequence that are identical with the amino acid residues in the specific polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or Megalign (DNASTAR) software. Those skilled in the art can determine appropriate parameters for measuring alignment, including any algorithms needed to achieve maximal alignment over the full length of the sequences being compared.

Percent amino acid sequence identity values may also be obtained as described below by using the WU-BLAST-2 computer program (Altschul et al., Methods in Enzymology 266:460-480 (1996)). Most of the WU-BLAST-2 search parameters are set to the default values. Those not set to default values, i.e., the adjustable parameters, are set with the following values: overlap span=1, overlap fraction=0.125, word threshold (T)=11, and scoring matrix=BLOSUM62. When WU-BLAST-2 is employed, a % amino acid sequence identity value is determined by dividing (a) the number of matching identical amino acid residues between the amino acid sequence of the polypeptide of interest having a sequence derived from the native polypeptide and the comparison amino acid sequence of interest (i.e., the sequence against which the polypeptide of interest is being compared which may be a variant polypeptide) as determined by WU-BLAST-2 by (b) the total number of amino acid residues of the polypeptide of interest. For example, in the statement “a polypeptide comprising an the amino acid sequence A which has or having at least 80% amino acid sequence identity to the amino acid sequence B”, the amino acid sequence A is the comparison amino acid sequence of interest and the amino acid sequence B is the amino acid sequence of the polypeptide of interest.

Percent amino acid sequence identity may also be determined using the sequence comparison program NCBI-BLAST2 (Altschul et al., Nucleic Acids Res. 25:3389-3402 (1997)). NCBI-BLAST2 uses several search parameters, wherein all of those search parameters are set to default values including, for example, unmask=yes, strand=a11, expected occurrences=10, minimum low complexity length=15/5, multi-pass e-value=0.01, constant for multi-pass=25, dropoff for final gapped alignment=25 and scoring matrix=BLOSUM62.

Isolated nucleic acids encoding the polypeptide of the invention, and having a sequence which differs from a nucleotide sequence shown in SEQ ID NO: 2 or 4 due to degeneracy in the genetic code are also within the scope of the invention. Such nucleic acids encode functionally equivalent proteins but differ in sequence from the sequence of SEQ ID NO: 2 or 4 due to degeneracy in the genetic code. Degeneracy means that a number of amino acids are designated by more than one triplet. DNA sequence polymorphisms that do lead to changes in the amino acid sequences of a protein will also exist within a population. Any and all such nucleotide variations and resulting amino acid polymorphisms are within the scope of the invention.

Other hydrolytic enzymes can be added to assist the breakdown of the lignocellulosic material. These enzymes can include but are not limited to; proteases, lipases, amylases, mananases, glucanases, pectinases, arabinofuranosidases and laccases.

The enzymes should be added to the slurry of lignocellulose or mixed in such as way as to ensure that the enzymes are evenly distributed throughout the slurry.

The slurry should then be warmed to a temperature that allows optimal activity of the enzymes, usually from 20° C. and 80° C. Preferably the slurry should be warmed to 50° C. A preferred embodiment of this invention is to incubate the enzymes and lignocellulose mixture at ambient temperatures to avoid the need for heating.

Samples can be taken from the reaction at regular intervals and the concentration of soluble sugars measured by a suitable method to monitor the progress of the reaction.

The enzymatic hydrolysis reaction is allowed to proceed for until the level of soluble sugars is no longer increasing. At least 30% of the total carbohydrate isolatable from the lignocellulosic material should have been converted to soluble sugars suitable for use as a fermentation feedstock.

Preferably, the reaction will be allowed to proceed for approximately 16 to 24 hours.

A preferred embodiment of this invention is the use of higher dosages of enzymes so that the reaction is completed in less than 4 hours.

After the hydrolysis reaction is complete the liquor containing soluble sugars can be separated from the remaining insoluble solids by settling, decanting, centrifugation or filtration.

Where the hydrolysed lignocellulosic material is to be used as a feedstock for simultaneous saccharification and fermentation there is no need to separate the liquor from the insoluble residue.

The invention will be further described with reference to the following examples and which are only exemplary and also by reference to the accompanying figures wherein:

FIG. 1 identifies the amino acid sequence of NsFaeD (SEQ ID No. 1) and the DNA sequence encoding it (SEQ ID No. 2).

FIG. 2 identifies the amino acid sequence of TsFaeC (SEQ ID No. 3) and the DNA sequence encoding it (SEQ ID No. 4).

### EXAMPLES

### Example 1

### Extraction of Soluble Sugars from Wheat Bran Using a Recombinant Type D Feruloyl Esterase from Neurspora crassa (NsFAED)

Wheat bran was milled to give particles less than 4 mm diameter. 300 g of the milled wheat bran was added to 1700 g of water and mixed to form a homogenous slurry. The pH was adjusted to pH 5.0 by the addition of 1N hydrochloric acid. Three hundred units of a recombinant type D feruloyl esterase from Neurospora crassa (NsFAED) and 10 ml of Depol 112L, (a commercial preparation available from Biocatalysts Ltd), which contains 800 units per g of cellulase and 4000 units per g of xylanase from Trichoderma sp., were added to the slurry and mixed to ensure even distribution of the enzyme. The slurry was then warmed to 50° C. after which the reaction was left to proceed for 24 hours. The hydrolysed lignocellulose slurry was centrifuged at 1000 g in a Beckman centrifuge to separate the liquor from the insoluble residue.

The concentration of soluble sugars in the liquor was measured using a dinitrosalicylic acid (DNS) assay (Miller, G. L. 1959) (refer Table 1). A control was also run with no FAED.

TABLE 1
Concentration of soluble sugars in liquor.

| | | | Total | |
|---|---|---|---|---|
| | | Glucose | glucose | |
| | | equivalents | equivalents | Glucose equivalents |
| | | in liquor | in | released per gram of |
| Substrate | Enzyme | (mg/ml) | liquor (mg) | wheat bran (mg/g) |
| Wheat | D112L | 5.01 | 9271.4 | 30.9 |
| bran | | | | |
| Wheat | D112L + | 5.45 | 10,082.5 | 33.6 |
| bran | FAE D | | | |

### Example 2

### Extraction of Soluble Sugars from Grass Using Recombinant Type D Feruloyl Esterase from Neurospora crassa (NsFAED) and Recombinant Type C from Taloromyces stiputatus (TsFAEC)

Stems from the biomass crop Miscanthus sp. were chopped in a blender to give chunks less than 4 mm long. 300 g of the chopped stems were added to 1700 g of water and the pH was adjusted to pH 5.0 by the addition of 1N hydrochloric acid. The slurry of chopped stems was then boiled for 15 minutes. After boiling the mixture was allowed to cool to 50° C.

Three hundred units of recombinant type D feruloyl esterase from Neurospora crassa (NsFAED), 300 units of recombinant type C feruloyl esterase from Taloromyces stiputatus (TsFAEC) and 20 ml Depol 692L (a commercial preparation from Biocatalysts Ltd), which contains 800 units per g of cellulase and >600 units per g of xylanase from Trichoderma sp. and 535 units/g of polygalacturonase from Aspergillus sp. were added to the slurry and mixed to ensure even distribution of the enzyme. The reaction mixture was then incubated at 50° C. for 24 hours.

The mixture containing hydrolysed lignocellulose was then centrifuged at 1000 g in a Beckman centrifuge after which the liquor was separated from the insoluble residue. The concentration of soluble sugars in the liquor was measured using a DNS assay (Miller, G. L. 1959) (refer Table 2). A control was also run with no FAE C/D.

TABLE 2
Concentration of soluble sugars in liquor.

| | | | | Glucose |
|---|---|---|---|---|
| | | | Total | equivalents |
| | | Glucose | glucose | released per gram |
| | | equivalents | equivalents | of |
| | | in liquor | in | Miscanthus sp. |
| Substrate | Enzyme | (mg/ml) | liquor (mg) | (mg/g) |
| Miscanthus | D692L | 6.27 | 11385.5 | 37.95 |
| sp. | | | | |
| Miscanthus | D692L + | 7.67 | 13915.6 | 46.38 |
| sp. | FAE | | | |
| | C & D | | | |

### Example 3

### Extraction of Soluble Sugars from Wheat Bran Using Depol 112L with and Without Additional Ferulic Acid Esterases C (TsFAEC) & D (NsFAED)

Wheat bran (23 g) was added to sodium acetate buffer (400 ml of 50 mM, pH 5.0) and mixed to form an homogenous slurry. The slurry was split into 2×200 ml parts and to one part was added Depol 112L (2 ml, a high xylanase Trichoderma cellulase from Biocatalysts Ltd.), was added to the slurry and mixed to ensure even distribution of the enzyme. To the second 200 ml part was added Depol 112L (2 ml) plus an enzyme preparation containing a mixture of recombinant FAE C (from Taloromyces stiputatus cloned and expressed in Pichia pastoris) & D (from Neurospora crassa cloned and expressed in Pichia pastoris) (combined FAE total 50 units). The slurries were warmed to 50° C. and enzyme hydrolysis was carried out under agitation for 24 hours at a temperature of 50° C. The hydrolysed lignocellulose slurry was filtered to separate the liquor from the insoluble residue. The concentration of soluble sugars in the liquor was measured using a DNS assay (Miller, G. L. 1959) (refer Table 3). A control was also run with no FAE C/D.

TABLE 3
Concentration of soluble sugars in liquor.

| | | | Total | |
|---|---|---|---|---|
| | | Glucose | glucose | |
| | | equivalents | equivalents | Glucose equivalents |
| | | in liquor | in | released per gram of |
| Substrate | Enzyme | (mg/ml) | liquor (mg) | wheat bran (mg/g) |
| Wheat | D112L | 7.32 | 1331.48 | 115.78 |
| bran | | | | |
| Wheat | D112L + | 11.59 | 1702.94 | 148.08 |
| bran | FAE | | | |
| | C & D | | | |

### Example 4

### Extraction of Soluble Sugars from Wheat Bran Using D112L (a Commercial Enzyme Preparation from Biocatalysts Ltd.) with and without Additional Ferulic Acid Esterases A & B

Wheat bran (23 g) was added to sodium acetate buffer (230 ml of 50 mM, pH 5.0) and mixed to form an homogenous slurry. The slurry was split into 2×115 ml parts and to one part was added Depot 112L (2 ml, a high xylanase Trichoderma cellulase from Biocatalysts Ltd.), was added to the slung and mixed to ensure even distribution of the enzyme. To the second 115 ml part was added Depot 112L (2 ml) plus an enzyme preparation containing a mixture of FAE A & FAE B (Depol 740L from Biocatalysts L:imited combined FAE total 36 units). The slurries were incubated at 50° C. and enzyme hydrolysis was carried out under agitation for 24 hours at a temperature of 50° C. The hydrolysed lignocellulose slurry was filtered to separate the liquor from the insoluble residue. The concentration of soluble sugars in the liquor was measured using a DNS assay (Miller, G. L. 1959) (refer Table 4). A control was also run with no FAE A/B.

TABLE 4
Concentration of soluble sugars in liquor.

| | | | Total | |
|---|---|---|---|---|
| | | Glucose | glucose | |
| | | equivalents | equivalents | Glucose equivalents |
| | | in liquor | in | released per gram of |
| Substrate | Enzyme | (mg/ml) | liquor (mg) | wheat bran (mg/g) |
| Wheat | D112L | 14.68 | 1372.24 | 119.32 |
| bran | | | | |
| Wheat | D112L + | 19.71 | 1931.69 | 167.97 |
| bran | FAE | | | |
| | A + B | | | |

### Example 5

### Extraction of Soluble Sugars from ‘Dried’ Miscanthus sp Using Depol 40L (Biocatalysts Ltd.) with and without Additional Ferulic Acid Esterases A & B

The biomass crop Miscanthus sp. was chopped in a blender to give chunks less than 20 mm long. 11.5 g of the ‘dried’ Miscanthus sp. was added to 230 ml of 50 mM sodium acetate buffer, pH 5.0. The slurry of blended Miscanthus sp. was then boiled for 30 minutes. After boiling, the mixture was allowed to cool to 50° C. The slurry was split into 2×115 int parts. To one part Depot 40L (1 ml, a high pectinase cellulase product from Biocatalysts Ltd.), was added to the slurry and mixed to ensure even distribution of the enzyme. To the second 115 ml part was added Depol 40L (1 ml) plus an enzyme preparation containing a mixture of FAE A & FAE B (Depot 740L from Biocatalysts combined FAE total 36 units). The slurries were warmed to 50° C. and enzyme hydrolysis was carried out under agitation for 24 hours at a temperature of 50° C. The hydrolysed lignocellulose slurry was filtered to separate the liquor from the insoluble residue. The concentration of soluble sugars in the liquor was measured using a DNS assay (Miller, G. L. 1959) (refer Table 5). A control was also run with no FAE A/B.

TABLE 5
Concentration of soluble sugars in liquor.

| | | | | Glucose |
|---|---|---|---|---|
| | | | Total | equivalents |
| | | Glucose | glucose | released per gram |
| | | equivalents | equivalents | of ‘dried’ |
| | | in liquor | in | Miscanthus sp. |
| Substrate | Enzyme | (mg/ml) | liquor (mg) | (mg/g) |
| Miscanthus | D40L | 2.73 | 225.6 | 39.23 |
| sp. | | | | |
| Miscanthus | D40L + | 3.61 | 308.67 | 53.68 |
| sp. | FAE A & | | | |
| | B | | | |

### Example 6

### Extraction of Soluble Sugars from Fresh Un-Dried Miscanthus sp Using Depol 40L (Biocatalysts Ltd.)

The biomass crop Miscanthus sp. was chopped in a blender to give chunks less than 20 mm long. 11.5 g of the fresh Miscanthus sp. was added to 230 ml of 50 mM sodium acetate buffer, pH 5.0. The slurry of blended Miscanthus sp. was then boiled for 30 minutes. After boiling, the mixture was allowed to cool to 50° C. The slurry was split into 2×115 ml parts. To one part Depol 40L (1 ml, a high pectinase cellulase product from Biocatalysts Ltd.), was added to the slurry and mixed to ensure even distribution of the enzyme. To the second 115 ml part was added Depol 40L (1 ml) plus an enzyme preparation containing a mixture of recombinant FAE C (from Taloromyces cloned and expressed in Pichia pastoris) & recombinant FAE D (from Neurospora cloned and expressed in Pichia pastoris) (combined FAE total 50 units). The slurries were warmed to 50° C. and enzyme hydrolysis was carried out under agitation for 24 hours at a temperature of 50° C. The hydrolysed lignocellulose slurry was filtered to separate the liquor from the insoluble residue. The concentration of soluble sugars in the liquor was measured using a DNS assay (Miller, G. L. 1959) (refer Table 6). A control was also run with no FAE C/D.

TABLE 6
Concentration of soluble sugars in liquor.

| | | | | Glucose |
|---|---|---|---|---|
| | | | Total | equivalents |
| | | Glucose | glucose | released per gram |
| | | equivalents | equivalents | of fresh |
| | | in liquor | in | Miscanthus sp. |
| Substrate | Enzyme | (mg/ml) | liquor (mg) | (mg/g) |
| Miscanthus | D40L | 2.11 | 206.29 | 35.88 |
| sp. | | | | |
| Miscanthus | D40L + | 2.90 | 290.37 | 50.50 |
| sp. | FAE C & | | | |
| | D | | | |

### REFERENCES

US Department of Agriculture (2002) Agricultural Research. vol 50(4) US Department of Agriculture, Washington, D.C., p 23.
Akin D. E., Morrison W. H., Rigsby L. L., Barton F. E., Himmelsbach D. S., and Hicks K. B. (2006) Con Stover Fractions and Bioenergy. Applied Biochemistry and Biotechnology. 129/1-3:104-116
Crepin V. F., Faulds C. B., and Connerton I. F. (2003) Production and characterisation of the Talaromyces stipitatus feruloyl esterase FAEC in Pichia pastoris: identification of the necleophile serine. Protein Expression and Purification 29:176-184.
Crepin V. F., Faulds C. B., and Connerton I. F. (2004a) Functional classification of the microbial feruloyl esterases. Applied Microbiology and Biotechnology. 63: 647-652
Crepin V. F., Faulds C. B., and Connerton I. F. (2004b) Identification of a type-D feruloyl esterase from Neurospora crassa. Applied Microbiology and Biotechnology. 63:567-70.
Faulds C. B., Zanichelli D., Crepin V. F., Connerton I. F., Juge N., Bhat M. K and Waldron K. W. (2003) Specificity of feruloyl esterases for water extractable and water unextractable feruloylated polysaccharides: influence of xylanases. Journal of Cereal Science 38:281-288.
Shin, H-D., McCledon S., Le T., Taylor F. and Chen R. R. (2006) A complete enzymatic recovery of ferulic acid from corn residues with extracellular enzymes from Neosartorya spinosa NRRL185. Biotechnology and Bioengineering 95:1108-1115.
Martin C., Galbe M, Wahlbom C. F, Hahn-Hagerdal B. and Jonsson L. J. (2002) Ethanol production from enzymatic hydrolysates of sugarcane bagasse using recombinant xylose—utilising Saccharomyces cervisiae. Enzyme and Microbial Technology 31:274-282.
McMillian J. D. (1994) In: enzymatic Conversion of Biomass for fuels Production. Himmel M. E. Baker J. O., and Overend R. P. (eds.) American Chemical Society, Washington, D.C., pp 292-324.
Ralph J., Grabber J. H. and Hatfield R. D. (1995) Lignin-ferulate cross links in grasses—active incorporation of ferulate polysaccharide esters into rye grass lignins. Carbohydrate Research 275:167-178.
Tabka M. G., Herpoel-Gimbert I., Monod F., Asther M. and Sigoillot J. C. (2006) Enzymatic saccharification of wheat straw for bioethanol production by a combined cellulase xylanase and feruloyl esterase treatment. Enzyme and Microbial Technology 39:897-902.
Williamson G., Kroon P. A. and Faulds C. B. (1998) Hairy plant polysaccharides: a close shave with microbial esterases. Microbiology 144:2011-2023.
Miller G. L. (1959) Use of dinitrosalicylic acid reagent for determination of reducing sugar. Analytical Chemistry 31, 426.

1-44. (canceled)

45. Use of an enzyme preparation comprising type C feruloyl esterase or type D feruloyl esterase in the manufacture of biofuels from plant cell wall materials.

46. Use of an enzyme preparation according to claim 45, wherein the use further comprises use of one or more plant cell wall-degrading enzymes selected from cellulase, xylanase, laccase, pectinase, glucanase, mannanase, amylase and arabinofuranosidase.

47. The use according to claim 45, wherein the feruloyl esterase is a mixture of type C feruloyl esterase and type D feruloyl esterase.

48. The use according to claim 45, wherein the feruloyl esterase is a mixture of type C feruloyl esterase and/or type D feruloyl esterase with type B feruloyl esterase and/or type A feruloyl esterase.

49. The use according to claim 45, wherein the feruloyl esterase is a wild-type or recombinant feruloyl esterase.

50. The use according to claim 49, wherein the recombinant feruloyl esterase is a recombinant type D feruloyl esterase which is a polypeptide with a sequence as shown in SEQ ID NO: 1, or a polypeptide having at least 80% sequence homology with a sequence as shown in SEQ ID NO: 1.

51. The use according to claim 49, wherein the recombinant feruloyl esterase is a recombinant type D feruloyl esterase encoded by the nucleic acid sequence of SEQ ID NO: 2.

52. The use according to claim 49, wherein the recombinant feruloyl esterase further includes a recombinant type C feruloyl esterase which is a polypeptide with a sequence as shown in SEQ ID NO: 3, or a polypeptide having at least 80% sequence homology with a sequence as shown in SEQ ID NO: 3.

53. The use according to claim 49, wherein the recombinant type C feruloyl esterase is encoded by the nucleic acid sequence of SEQ ID NO: 4 and the recombinant type D feruloyl esterase is encoded by the sequence of SEQ ID NO: 2.

54. A method for manufacturing biofuels from plant cell wall materials by converting lignocellulosic materials in said plant cell walls to sugars suitable for use as a fermentation feedstock, which method comprises:
(i) contacting said plant cell wall material with an enzyme preparation comprising type C feruloyl esterase or type D feruloyl esterase and plant cell wall-degrading enzymes; and
(ii) separating any soluble sugars therefrom for bioconversion to biofuel.

55. The method according to claim 54, wherein said plant cell wall-degrading enzyme further comprises one or more of a cellulase, xylanase, laccase, pectinase, glucanase, mannanase, amylase and arabinofuranosidase.

56. The method according to claim 54, wherein the feruloyl esterase is a type D feruloyl esterase.

57. The method according to claim 54, wherein the feruloyl esterase is a mixture of type C feruloyl esterase and type D feruloyl esterase.

58. The method according to claim 54, wherein the feruloyl esterase is a mixture of type C feruloyl esterase and/or type D feruloyl esterase with type B feruloyl esterase and/or type A feruloyl esterase.

59. The method according to claim 54, wherein the feruloyl esterase is a wild-type or recombinant feruloyl esterase.

60. The method according to claim 59, wherein the recombinant feruloyl esterase is a recombinant type D feruloyl esterase which is a polypeptide with a sequence as shown in SEQ ID NO: 1, or a polypeptide having at least 80% sequence homology with a sequence as shown in SEQ ID NO: 1.

61. The method according to claim 59, wherein the recombinant feruloyl esterase is a recombinant type D feruloyl esterase encoded by the nucleic acid sequence of SEQ ID NO: 2.

62. The method according to claim 60, wherein the recombinant feruloyl esterase further includes a recombinant type C feruloyl esterase which is a polypeptide with a sequence as shown in SEQ ID NO: 3, or a polypeptide having at least 80% sequence homology with a sequence as shown in SEQ ID NO: 3.

63. The method according to claim 62, wherein the recombinant type C feruloyl esterase is encoded by the nucleic acid sequence of SEQ ID NO: 4 and the recombinant type D feruloyl esterase is encoded by the sequence of SEQ ID NO: 2.

64. A slurry for use in the manufacture of biofuels prepared by converting lignocellulosic materials in plant cell walls to sugars using an enzyme preparation comprising type C feruloyl esterase or type D feruloyl esterase, and optionally plant cell-wall degrading enzymes.
